(19) United States
(12) Patent Application Publication
(10) Pub. No.: US 2020/0289316 A1
(43) Pub. Date: Sep. 17, 2020

(54) DEVICE AND METHOD FOR THE ADJUSTMENT OF TEMPERATURE

(71) Applicant: LAUDA DR. R. WOBSER GMBH & CO. KG, Lauda-Königshofen (DE)

(72) Inventor: Mathias Eller, Altertheim (DE)

(73) Assignee: LAUDA DR. R. WOBSER GMBH & CO. KG, Lauda-Königshofen (DE)

(21) Appl. No.: 16/819,274

(22) Filed: Mar. 16, 2020

(30) Foreign Application Priority Data
Mar. 15, 2019 (DE) 102019106713.8

(51) Int. Cl. A61F 7/00; A61M 1/16

(52) U.S. Cl. CPC A61F 7/0085; A61M 1/1698; A61F 2007/0054; A61M 2205/366; A61M 1/1629

(57) ABSTRACT

Device (100, 201) for the adjustment of temperature, especially for the extracorporeal adjustment of the temperature of patients, with a transfer of heat between a fluid and a transfer medium, including a temperature-adjustment body (203) having a primary side which provides a guide for the fluid and having an application side which provides a surface (219) for contacting the temperature-adjustment body (203) with the transfer medium; a cover (221) which is attached to the temperature-adjustment body (203) and which forms a cavity with the temperature-adjustment body (203) on the application side, through which cavity the transfer medium can flow; the cover (221) being attached to the temperature-adjustment body (203) in a detachable manner; and flow-guiding structures (217) for guiding the transfer medium being arranged on the temperature-adjustment body (203) and/or on the cover (221).

### BACKGROUND OF THE INVENTION

The invention relates to a device and a method for the adjustment of temperature, especially for the extracorporeal adjustment of the temperature of patients and for the extracorporeal adjustment of the temperature of body fluids.

Methods and devices which use or comprise heat exchangers are known for the extracorporeal adjustment of the temperature of patients. In many cases, a liquid based on water as medium for the transfer of heat or cold is used for the heat exchangers.

However, hitherto known solutions from the prior art have restrictions in relation to the transferable heat output, especially in relation to a housing volume of the heat exchanger, in relation to the lasting fulfillment of hygiene requirements, or they are just complicated to clean.

### SUMMARY OF THE INVENTION

It is an object of the invention to provide an improved device for the adjustment of temperature, especially for the extracorporeal adjustment of the temperature of patients or of body fluids. In particular, hygiene should be improved or the transferable heat output, especially in relation to an assembly space of the device, should be improved or the construction should be simplified or easier to clean or otherwise facilitate the observance of hygiene standards.

According to one aspect of the invention, what is provided is a device for the adjustment of temperature, especially for the extracorporeal adjustment of the temperature of patients or of body fluids, with a transfer of heat between a fluid and a transfer medium, comprising a temperature-adjustment body having a primary side which provides a guide for the fluid and having an application side which provides a surface for contacting the temperature-adjustment body with the transfer medium; a cover which is attached to the temperature-adjustment body and which forms a cavity with the temperature-adjustment body on the application side, through which cavity the transfer medium can flow; the cover being attached to the temperature-adjustment body in a detachable manner; and flow-guiding structures for guiding the transfer medium being arranged on the temperature-adjustment body or on the cover, which flow-guiding structures typically stretch from the surface of the application side up to a cover plate of the cover when the cover is attached to the temperature-adjustment body.

According to a further aspect of the invention, what is provided is a method for the adjustment of temperature, especially for the extracorporeal adjustment of the temperature of patients or of body fluids, using a temperature-adjustment body and two covers, especially in accordance with a device according to any of the embodiments described herein, comprising attaching the first cover to the temperature-adjustment body, using the device to adjust the temperature of the transfer medium, detaching the first cover from the temperature-adjustment body, attaching the second cover to the temperature-adjustment body, using the device to adjust the temperature of the transfer medium.

Typical temperature-adjustment bodies are, on the primary side of the temperature-adjustment body, connected to a primary loop in which a fluid is accommodatable. Typically, the fluid is a liquid or a gas or a gas/liquid mixture that is suitable for storing and transferring heat, for example water, vegetable oil, ammonia, carbon dioxide, hydrocarbons, hydrofluorocarbons, inorganic coolants or air. The term heat or transfer of heat is also understood herein to mean cold or the transfer of cold. The terms “heat” and “transfer of heat” are understood herein in a physical sense.

Typically, the temperature-adjustment body has, on the application side of the temperature-adjustment body, a surface for contacting the temperature-adjustment body with the transfer medium and for transferring heat between the temperature-adjustment body and the transfer medium. The transfer medium is typically accommodated in a secondary loop. Typically, the transfer medium is a liquid. In typical embodiments, a body fluid is extracorporeally temperature-adjusted using the secondary loop. In typical embodiments, the transfer medium comprises at least 90% by weight or at least 95% by weight of water, especially water for injection (WFI). In a typical embodiment, the transfer medium is a physiological or isotonic medium. Typically, the transfer medium is sterile or largely free of pathogens, for example fewer than 1000 or not more than 100 colony-forming aerobic pathogens per liter. Physiological or isotonic media are, for example, a 0.9% solution containing common salt (sodium chloride, NaCl) or a Ringer's solution.

In typical embodiments of the device, the temperature-adjustment body forms, on the application side of the temperature-adjustment body, a cavity with the cover attached to the temperature-adjustment body. Typically, the cavity, with the exception of ports for supplying and removing the transfer medium, is closed in a liquid-tight manner.

Typically, the cavity is enclosed by the surface of the application side of the temperature-adjustment body, which surface is intended for contacting with the transfer medium, by a cover plate of the cover that is facing the surface of the application side of the temperature-adjustment body, especially by one that is at least substantially parallel to the surface, and by an edge plate which especially extends between the edges of the cover plate of the cover and the surface of the application side of the temperature-adjustment body. Typically, the edge plate is realized as an integral part of the cover. Typically, a cover comprising a cover plate and an edge plate is placed as a hood onto the surface of the application side of the temperature-adjustment body. In further embodiments, the edge plate is realized as part of the temperature-adjustment body. In further embodiments, the cover plate is arched or curved relative to the surface of the application side of the temperature-adjustment body.

In typical embodiments, the flow-guiding structures are produced in an integral manner with the part of the temperature-adjustment body providing the surface of the application side or the flow-guiding structures are later mounted on the surface of the application side of the temperature-adjustment body. In further typical embodiments, the flow-guiding structures are produced as part of the cover or later mounted on the cover plate of the cover. In typical embodiments, at least some of the flow-guiding structures extend between the surface of the application side and the cover plate of the cover over at least 50%, typically over at least 70% or over at least 90%, of the distance between the surface of the application side and the cover plate of the cover.

In typical embodiments, the temperature-adjustment body and the cover form, owing to the flow-guiding structures, channels through which the transfer medium can flow. The cavity formed by the temperature-adjustment body and the cover is typically divided into channels by flow-guiding structures, realized as ribs for example. Typically, channels run in a parallel or meandering manner. In typical embodiments, the flow-guiding structures guide the transfer medium in parallel or in series through at least 5, especially through at least 10 or at least 20 channels. Flow-guiding structures realized as ribs are typically realized as straight or as wavy ribs. Typically, the ribs stretch from the surface of the temperature-adjustment body up to the cover plate of the cover, the result being that a flow of transfer medium between two adjacent channels can be at least substantially prevented.

In typical embodiments, the flow-guiding structures guide a flow of the transfer medium in the cavity formed by the temperature-adjustment body and the cover in a parallel, antiparallel or meandering manner at least in part. By configuring the inner side, especially the side of the cover that is facing the transfer medium, it is possible to provide various flow variants. All channels parallel, some channels parallel or said channels parallel at least in part meandering or an individual meandering channel.

In typical embodiments, the temperature-adjustment body or the cover comprises swirling structures for swirling the transfer medium. Swirling structures are typically upstream or downstream of flow-guiding structures or arranged in channels formed by flow-guiding structures. Swirling structures comprise, for example, cylindrical structures which extend between the cover plate of the cover and the surface of the application side of the temperature-adjustment body. Swirling structures can increase the heat transfer between temperature-adjustment body and transfer medium compared to an unswirled flow guide or can improve the flow through channels.

In typical embodiments, the temperature-adjustment body comprises ports for supplying and removing the fluid and tubes for guiding the fluid. Typically, the ports are arranged on the primary side of the temperature-adjustment body. Typically, the tubes are connected to chambers in the temperature-adjustment body or are interrupted by the chambers. The chambers can be provided especially for the uniform charging of typically parallel tubes with the fluid. Typically, the fluid flows through the pipes in parallel or in series.

Typically, the temperature-adjustment body or the cover comprises ports for supplying and removing the transfer medium. In a typical embodiment, the ports are arranged on corners of a cover that are facing one another. Typically, the ports are designed such that an at least substantially complete emptying of a cavity filled with transfer medium is possible before the detachment of the cover.

In typical embodiments, the temperature-adjustment body is multi-piece. The temperature-adjustment body is typically produced by connecting one or more metal blocks, especially by connecting multiple machined metal blocks, to ports for supplying and removing the transfer medium or the fluid, to tubes for guiding the fluid, to flow-guiding structures or to swirling structures.

In typical embodiments, the temperature-adjustment body is produced especially as one piece by machining a metal block or by an additive manufacturing process such as, for example, a metal 3D printing process. Further machining steps or further parts or blocks may be present. In typical embodiments, the flow-guiding structures are produced on the application side of the temperature-adjustment body or the tubes for guiding the fluid are produced on the primary side of the temperature-adjustment body by a material-removal process for a metal block of the temperature-adjustment body. Typical material-removal processes used are, for example, milling or erosion.

In typical embodiments, the temperature-adjustment body comprises at least one first metal block, and a second metal block. The first metal block and the second metal block are typically connected to one another by a joining process, especially soldered or screwed or welded to one another, for example by diffusion welding. Typically, grooves are worked in the first metal block or in the second metal block on the side facing the other metal block using a material-removal process.

The grooves are typically realized such that they form the tubes or chambers for guiding the fluid. In typical exemplary embodiments, the tubes can also be referred to as fluid channels. In embodiments, the grooves are realized for the accommodation of insertable tubes, for example of microchannels. In further embodiments, the grooves are free from further fittings, for example free from microchannels.

Typically, the temperature-adjustment body comprises a metal block which provides, on the application side of the temperature-adjustment body, a surface for contacting the temperature-adjustment body with the transfer medium. In a typical embodiment, the flow-guiding structures are produced on the surface by milling or erosion of the metal block. In further typical embodiments, the flow-guiding structures are produced as separate parts and later connected to the metal block by a joining process, especially soldered or welded to the metal block.

In typical embodiments, the device comprises a temperature sensor which can be connected to a temperature display or a control electronics unit. Typically, a temperature sensor for determining the temperature of the fluid is mounted on the temperature-adjustment body. Typically, a temperature sensor for determining the temperature of the transfer medium is mounted on the temperature-adjustment body or on the cover.

In typical embodiments, the cover comprises a seal which seals the cavity at the boundary between the temperature-adjustment body and the cover attached to the temperature-adjustment body. Typically, the cover comprises at least one of the following seals: a labyrinth seal, a gap seal, a seal composed of a sealing material such as, for example, plastic, metal or organic or inorganic materials, a flat seal, or a cohesive seal. Typically, the seal is realized on the cover, especially on the temperature-adjustment body-facing edge of the edge plate of the cover. In embodiments, the seal is realized on the application side of the temperature-adjustment body or as a separate element.

Typically, the cover is insulated against heat losses from the cavity toward the outside. The cover can comprise an insulating multichamber system, especially with negative pressure in the multichamber system, an insulating multilayer system or an insulating coating.

In typical embodiments, the cover is detachable from the temperature-adjustment body or especially repeatedly attachable to the temperature-adjustment body. Typically, the cover is detachable from the temperature-adjustment body without any damage. In a typical embodiment, the cover is repeatably detachable from the temperature-adjustment body. A cover realized so as to be detachable from the temperature-adjustment body can offer the advantage that typical devices according to the invention can be kept pathogen-free, especially by drying and cleaning the application side of the temperature-adjustment body with the cover detached.

In typical embodiments, the device for the adjustment of temperature comprises a connecting element for the undoable attachment of the cover to the temperature-adjustment body. The connecting element comprises especially a screw, an adhesive, a clamping means or a magnet. In further embodiments, the cover is attached to the temperature-adjustment body by means of negative pressure, in comparison with the environment, in the cavity formed by the temperature-adjustment body and the cover or by means of adhesion forces between the cover and the temperature-adjustment body, especially in addition to the attachment using a connecting means.

In typical embodiments, the temperature-adjustment body and the cover are made of different materials. Typically, the temperature-adjustment body is at least substantially made of metal. Typically, the temperature-adjustment body is composed of metal to an extent of at least 90%, or to an extent of at least 80%, or to an extent of at least 70%. These data are based on % by weight. In further typical embodiments, the temperature-adjustment body is composed of metal to an extent of less than 100% or composed of metal to an extent of less than 50%. If the temperature-adjustment body is multi-piece, it is also possible for parts of the temperature-adjustment body to be made of different materials. Typically, at least a metal block providing a surface for contacting the temperature-adjustment body with the transfer medium, or possibly flow-guiding structures arranged on the metal block, are made of particularly heat-conductive materials. Typical metals which can be used are aluminum alloys, especially those with a surface finish, brass or copper. Typical plastics encompass, for example, polymers such as PP or PE realized in various ways.

Typically, the cover is at least substantially made of a plastic or at least substantially made of glass. Typically, the cover is composed of plastic to an extent of at least 90%, or to an extent of at least 80%, or to an extent of at least 70%. These data are based on % by weight. In further typical embodiments, the cover is composed of plastic to an extent of less than 100% or composed of plastic to an extent of less than 50%. Typically, the cover is composed of glass to an extent of at least 90%, or to an extent of at least 80%, or to an extent of at least 70%. In further typical embodiments, the cover is composed of glass to an extent of less than 100% or composed of glass to an extent of less than 50%.

In typical embodiments, the cover is designed for single use. In the case of a single use, the cover is, for example, designed as a disposable article or one-time article. A reliable observance of hygiene standards can be achieved as a result. In particular, the cover intended for single use is not autoclavable. Typically, the cover is produced so as to be sterile or largely pathogen-free. In further embodiments, the cover is designed for multiple use, in particular constructed so as to be autoclavable. In this way, the cover can be used multiple times, and waste can be saved as a result.

In typical embodiments of a method for the adjustment of temperature, especially for the extracorporeal adjustment of the temperature of patients, a first cover is attached to a temperature-adjustment body. To use the device for the adjustment of the temperature of a first transfer medium, a primary loop providing a fluid is typically connected on the primary side of the temperature-adjustment body to ports for supplying and removing the fluid. Typically, a secondary loop providing the first transfer medium is connected on the application side of the temperature-adjustment body to ports for supplying and removing the first transfer medium. Typically, the tubes of the temperature-adjustment body are filled with the fluid or the fluid flows through them. Typically, the cavity formed by the temperature-adjustment body and the cover is filled with the first transfer medium and the first transfer medium flows through it.

In typical methods, the use of the device for the adjustment of the temperature of the first transfer medium comprises a vaporization of the fluid in the tubes of the temperature-adjustment body following a cooling requirement. Alternatively or additionally, what can be carried out in typical methods is a liquefaction of the fluid in the tubes following a heating requirement.

Typically, the first cover is detached from the temperature-adjustment body after the use of the device for the adjustment of the temperature of the first transfer medium, especially after the draining of the first transfer medium. Typically, the temperature-adjustment body, especially the application side of the temperature-adjustment body, is cleaned after the detachment of the first cover. The cleaning is typically done using sterilizing or disinfectant cleaning agents. In typical embodiments, the cleaning comprises a drying operation, especially by heating the temperature-adjustment body.

Typically, a second cover is then attached to the temperature-adjustment body. Typically, a secondary loop providing second transfer medium is connected on the application side of the temperature-adjustment body to ports for supplying and removing the second transfer medium. In typical embodiments of the method, the device is subsequently used for the adjustment of the temperature of the second transfer medium.

In typical embodiments, the first cover and the second cover are each disposed of after use. In further typical embodiments, the first cover and the second cover are cleaned or sterilized, for example autoclaved, after use and are reused.

Typical advantages of embodiments encompass, for example, the fact that the flow-guiding structures can be realized on the temperature-adjustment body or on the cover in an application-specific manner. Different temperature-adjustment tasks can be managed, not just an extracorporeal adjustment of the temperature of patients or of body fluids. It is also possible to adjust the temperature of other media. A further advantage of typical embodiments can be that the flow-guiding structures ensure a high degree of heat transfer between the primary side of the temperature-adjustment body, especially the fluid, and the transfer medium. One advantage of typical embodiments can be the undoable attachment of the cover and the good accessibility of the surface of the application side of the temperature-adjustment body. Typical embodiments offer the option of an efficient cleaning or of realizing the cover or other parts of the claimed device as disposable product.

### BRIEF DESCRIPTION OF THE DRAWINGS

Exemplary embodiments of the invention will be more particularly elucidated below with reference to drawings, where:

FIG. 1 shows a schematic structure of a system comprising a typical device for the adjustment of temperature, especially for the extracorporeal adjustment of the temperature of patients or for the extracorporeal adjustment of the temperature of body fluids;

FIG. 2 shows a schematic exploded view of a typical device; and

FIG. 3 shows a method in a typical embodiment.

### DESCRIPTION OF THE EXEMPLARY EMBODIMENTS SHOWN IN THE FIGURES

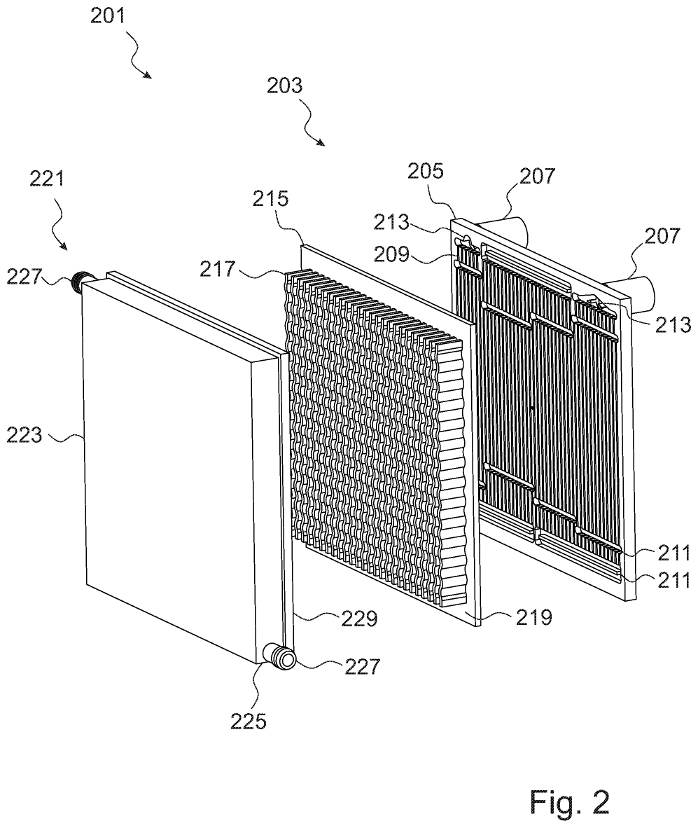

Typical exemplary embodiments of the invention will be described below, with sometimes the same reference signs being used for identical or similar parts and possibly not being explained again with each figure. The invention is not restricted to the typical embodiments described below. In some cases, not all features in question are provided with a reference sign for the sake of clarity, for example the grooves with the reference sign 209 in FIG. 2.

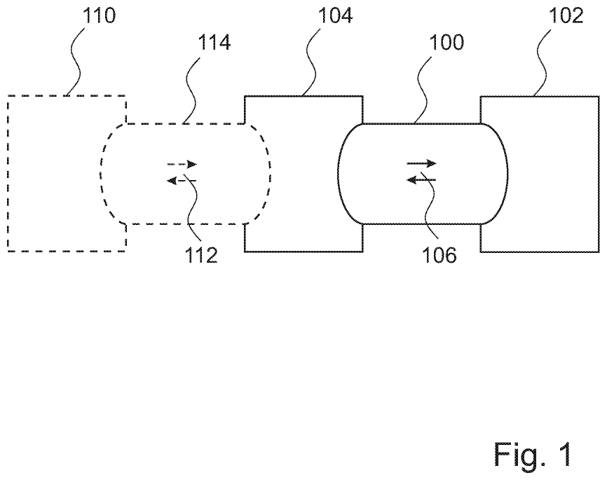

FIG. 1 shows schematically a device 100, which is a typical embodiment of the invention. Typically, the device 100 comprises a temperature-adjustment body and a cover attached to the temperature-adjustment body, the temperature-adjustment body having a primary side which provides a guide for a fluid of a primary loop 102 and an application side which provides a surface for contacting the temperature-adjustment body with a transfer medium of a secondary loop 104. The temperature-adjustment body of the device 100 allows the heat transfer 106 between a fluid of the primary loop 102 and a transfer medium of the secondary loop 104.

The wording “heat transfer” encompasses, as in general in this description, a transfer of heat in at least one arbitrary direction or in both directions, especially a heating or a cooling of the transfer medium.

The heat transfer is typically realized by the contact of the fluid with the temperature-adjustment body on the primary side of the temperature-adjustment body, by the heat transfer in the temperature-adjustment body, and by the contact of the transfer medium with the temperature-adjustment body on the application side of the temperature-adjustment body. In typical embodiments, the efficiency or precision of the heat transfer is increased by flow-guiding structures, especially by heat-conducting flow-guiding structures, compared to unguided flow.

The transfer medium, an isotonic 0.9% NaCl solution in the example depicted in FIG. 1, of the secondary loop 104 transfers heat to a heat exchanger 114, which is, for example, part of an oxygenator. In the heat exchanger 114, heat is transferred between the transfer medium of the secondary loop 104 and a body fluid or liquid 110, for example blood of a patient, as a result of a heat exchange 112.

Using typical devices, a patient or an animal, the blood circulation of which is guided through the oxygenator at least in part, is temperature-adjusted, i.e., cooled or heated.

FIG. 2 shows an exploded view of a typical device 201 for the adjustment of temperature, comprising a temperature-adjustment body 203 and a cover 221 attached to the temperature-adjustment body. In the exemplary embodiment in FIG. 2, the temperature-adjustment body 203 comprises a first metal block 205 and a second metal block 215, which are welded to one another.

Mounted on a primary side of the temperature-adjustment body 203 are primary ports 207 as ports for supplying and removing a fluid. In the embodiment shown, the primary ports 207 are mounted on the first metal block 205.

The temperature-adjustment body 203 contains tubes 209 for guiding the fluid. The primary ports 207 are connected to the tubes 209 via ducts 213. The ducts 213 are, as shown in FIG. 2 by way of example, milled through the first metal block 205.

The primary ports 207 of the temperature-adjustment body 203 are connected to one another by the tubes 209. In the embodiment in FIG. 2, the tubes 209 are connected to chambers 211 or are interrupted by the chambers 211. The chambers 211 are provided for the uniform charging of the parallel tubes 209 with the fluid.

In the exemplary embodiment in FIG. 2, grooves and indentations are milled or eroded into the first metal block 205. The grooves and indentations are covered by the second metal block 215 as a result of the connection of the first metal block 205 and thus closed to form tubes 209 or chambers 211.

The temperature-adjustment body 203 comprises flow-guiding structures 217 on a surface 219 of an application side of the temperature-adjustment body 203. In the exemplary embodiment in FIG. 2, the flow-guiding structures 217 are realized as wavy ribs. In the exemplary embodiment in FIG. 2, the flow-guiding structures 217 are milled out of the second metal block 215.

In embodiments, suitability for comprehensive cleaning is, for example, due to the possibility of detaching the cover and of sterilizing and drying the temperature-adjustment body. Typical embodiments comprise a temperature-adjustment body or cover at least substantially free from undercut structures. In particular, the ribs are realized without any undercuts on the cover or on the temperature-adjustment body.

The cover 221 of the device 201 is attached to the temperature-adjustment body 203 in a detachable manner. In the exemplary embodiment in FIG. 2, the cover 221 comprises a cover plate 223, which runs substantially parallel to the surface 219 of the application side of the temperature-adjustment body 203, an edge plate 225, which extends between the edge of the cover plate 223 and the surface 219 of the application side of the temperature-adjustment body 203, secondary ports 227 as ports for supplying and removing a transfer medium, and a connecting element 229 for attaching the cover 221 to the temperature-adjustment body 203.

The flow-guiding structures 217 of the exemplary embodiment in FIG. 2 are realized such that they extend from the surface 219 of the application side of the temperature-adjustment body 203 up to the cover plate 223 of the cover 221. The flow-guiding structures 217 form channels which run in parallel for guiding the transfer medium.

In the case of one use of the device 201 in the typical embodiment in FIG. 1, transfer medium flows through a first secondary port of the secondary ports 227 into the cavity formed by the temperature-adjustment body 203 and the cover 221, flows in parallel through the channels formed by the flow-guiding structures 217, and exits the cavity at a second secondary port of the secondary ports 227.

In the exemplary embodiment in FIG. 2, the cover 221 is inserted onto the temperature-adjustment body 203 in a detachable manner by means of the connecting element 229, which is realized as a strip-shaped clamping means on the edge plate 225. Typically, the cover 221 comprises a seal, which, for example, is arranged on the edge of the edge plate 225 that is facing the surface 219 of the temperature-adjustment body 203.

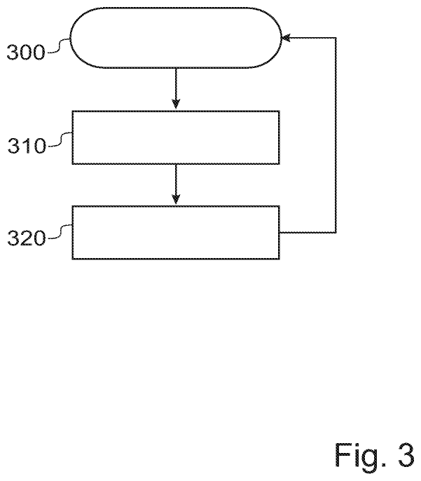

FIG. 3 shows schematically a typical method for the extracorporeal adjustment of the temperature of patients, with a transfer of heat between a fluid and a transfer medium. The method can, for example, be carried out using a typical embodiment of a device as elucidated in connection with FIGS. 1 and 2.

In the exemplary method in FIG. 3, in 300, a first cover is attached to a temperature-adjustment body in a detachable manner by means of a clamping means as a connecting element.

The undoable attachment is typically undoable without any damage. Typically, a first cavity formed by the temperature-adjustment body and the first cover as a result of the undoable attachment is sealed against an uncontrolled escape of transfer medium.

In 310, a device comprising the temperature-adjustment body and the first cover is used for the adjustment of the temperature of a first transfer medium in the device by means of the fluid guided through the temperature-adjustment body. The adjustment of temperature is effected by a vaporization of the fluid following a cooling requirement and a liquefaction of the fluid following a heating requirement. The heat output of the primary side is regulated as needed such that the desired temperature ensues in the secondary loop. In this connection, the temperature in the primary loop or the temperature in the secondary loop can in each case be higher or lower by a few degrees depending on requirements.

In 320, the first cover is detached from the temperature-adjustment body without any damage, especially after the draining of the first transfer medium from the first cavity.

Typically, after 320, the temperature-adjustment body, especially a surface of the temperature-adjustment body that was in contact with the first transfer medium, is cleaned. Typically, the cleaning comprises a sterilization and a drying of the temperature-adjustment body or of the surface of the temperature-adjustment body that was in contact with the first transfer medium. In a typical embodiment of the method, the first cover is disposed of. In further embodiments, the first cover is cleaned and sterilized for reuse.

The method then jumps back to 300 for the next patient, with use then being made of a second cover and a second transfer medium in the second run, meaning that any pathogens brought into the first transfer medium in the first run do not lead to a contamination. A third run and further runs of the method can take place for the adjustment of the temperature of further patients.

The repetition can also be done multiple times or in further exemplary embodiments with a cleaned and sterilized cover.

1. Device (100, 201) for the adjustment of temperature, especially for the extracorporeal adjustment of the temperature of patients and/or for the extracorporeal adjustment of the temperature of body fluids, with a transfer of heat between a fluid and a transfer medium, comprising
a temperature-adjustment body (203) having a primary side which provides a guide for the fluid and having an application side which provides a surface (219) for contacting the temperature-adjustment body (203) with the transfer medium;
a cover (221) which is attached to the temperature-adjustment body (203) and which forms a cavity with the temperature-adjustment body (203) on the application side, through which cavity the transfer medium can flow;
the cover (221) being attached to the temperature-adjustment body (203) in a detachable manner; and
flow-guiding structures (217) for guiding the transfer medium being arranged on the temperature-adjustment body (203) and/or on the cover (221).

2. Device according to claim 1, wherein the flow-guiding structures stretch from the surface (219) of the application side up to a cover plate (223) of the cover (221) when the cover (221) is attached to the temperature-adjustment body (203).

3. Device according to claim 1, wherein the temperature-adjustment body (203) and/or the cover (221) comprise swirling structures for swirling the transfer medium.

4. Device according to claim 1, wherein the temperature-adjustment body (203) comprises ports (207) for supplying and removing the fluid and tubes (209) for guiding the fluid.

5. Device according to claim 1, wherein the temperature-adjustment body (203) and/or the cover (221) comprise ports (227) for supplying and removing the transfer medium.

6. Device according to claim 1, wherein the temperature-adjustment body (203) is multi-piece.

7. Device according to claim 1, wherein the temperature-adjustment body (203) is produced especially as one piece by machining a metal block (205, 215) and/or by an additive manufacturing process.

8. Device according to claim 1, wherein the flow-guiding structures (217) are produced on the application side of the temperature-adjustment body (203) and/or the tubes (209) for guiding the fluid are produced on the primary side of the temperature-adjustment body (203) by a material-removal process for a metal block (205, 215) of the temperature-adjustment body (203).

9. Device according to claim 1, wherein the cover (221) comprises a seal which seals the cavity at the boundary between the temperature-adjustment body (203) and the cover (221) attached to the temperature-adjustment body (203).

10. Device according to claim 1, comprising a connecting element (229) for the undoable attachment of the cover (221) to the temperature-adjustment body (203).

11. Device according to claim 1, wherein the temperature-adjustment body (203) and the cover (221) are made of different materials.

12. Device according to claim 1, wherein the cover (221) is designed for single use.

13. Device according to claim 1, wherein the cover (221) is at least substantially made of a plastic or at least substantially made of glass.

14. Method for the adjustment of temperature using a temperature-adjustment body and two covers in accordance with a device according to claim 1, comprising
attaching (300) a first cover to the temperature-adjustment body,
using (310) the device to adjust the temperature of the transfer medium,
detaching (320) the first cover from the temperature-adjustment body,
attaching a second cover to the temperature-adjustment body,
using the device to adjust the temperature of the transfer medium.

15. Method according to claim 14, wherein the first cover and the second cover are each disposed of after use.

16. Method according to claim 14, wherein the method comprises a method for the extracorporeal adjustment of the temperature of patients and/or for the extracorporeal adjustment of the temperature of body fluids.
